(19) United States
(12) Patent Application Publication
(10) Pub. No.: US 2010/0069760 A1
(43) Pub. Date: Mar. 18, 2010

(54) METHODS AND APPARATUS FOR ANALYZING AND LOCALLY TREATING A BODY LUMEN

(75) Inventor: Jing Tang, Arlington, MA (US)

Correspondence Address: MILLS & ONELLO LLP, ELEVEN BEACON STREET, SUITE 605, BOSTON, MA, 02108, US

(73) Assignee: CorNova, Inc., Burlington, MA (US)

(21) Appl. No.: 12/561,756

(22) Filed: Sep. 17, 2009

Related U.S. Application Data
(60) Provisional application No. 61/097,578, filed on Sep. 17, 2008.

(51) Int. Cl. A61M 37/00; A61B 6/00

(52) U.S. Cl. 600/478; 604/510

(57) ABSTRACT

A method and apparatus for analyzing and treating internal lumens is provided. The apparatus includes a catheterized device integrating an optical probe and local treatment delivery system. The probe component includes fiber optic lines that can be used in conjunction with visible and/or near infrared spectroscopy to analyze various characteristics of tissues, including chemical, blood, and oxygen content, in order to locate those tissues associated with diseased lumens, to determine the best location for applying treatment, and to monitor treatment and its effects. Physically integrated with the probe component is a treatment component for delivering localized treatments including stem cells, antibiotics, gene therapy, neoplasty, and sclerosant drugs, etc. A control system coordinates operation of the catheter, including performing chemometric analysis with the use of model data, and for providing control and visual feedback to an operator.

### RELATED APPLICATIONS

This application claims the benefit of U.S. Patent Application No. 61/097,578, filed on Sep. 17, 2008, entitled “Method and Apparatus for Treating a Body Lumen,” the contents of which is incorporated herein in its entirety by reference.

This application is related to U.S. patent application Ser. No. 11/762,956, filed on 14 Jun. 2007, entitled “Method and Apparatus for Identifying and Treating Myocardial Infarction,” the contents of which is incorporated herein in its entirety by reference.

### FIELD OF THE INVENTION

This invention relates to methods and apparatus for identifying, localizing, and treating diseased or damaged internal tissues including tissue surrounding internal body lumens. The invention relates to, in particular, catheters having optical-probe and needle-injection assemblies.

### BACKGROUND OF THE INVENTION

Cardiovascular diseases and disorders are the leading cause of death and disability in all industrialized nations. Common conditions include blocked or stenotic coronary vessels such as those affected by the buildup of cholesterol-laden plaques that form due to atherosclerosis. Other conditions of vessels include the formation of blood clots (thrombosis) that can result in life-threatening events such as heart attacks or stroke.

Traditional techniques for treating diseased vessels include angioplasty, stenting, systemic drug therapy, and or coronary bypass, each of which can carry significant risks either during or after treatment. An angioplasty procedure (i.e., percutaneous transluminal angioplasty, or “PTA”) utilizes a flexible catheter with an inflation lumen to expand, under relatively high pressure, a balloon at the distal end of the catheter to expand a stenotic lesion. The procedures are now commonly used in conjunction with expandable tubular structures known as stents. An angioplasty balloon is often used to expand and permanently place the stent within the lumen. A risk with a conventional stent, however, is the reduction in efficacy of the stent due to the growth of the tissues surrounding the stent which can again result in the stenosis of the lumen, often referred to as restenosis. In recent years, new stents that are coated with a pharmaceutical agents, often in combination with a polymer, have been introduced and shown to significantly reduce the rate of restenosis. However, some studies suggest that these drug-eluting stents may increase the risk of blood clots and are often prescribed with life-long clot-inhibiting drug therapies. The clot-inhibiting therapies, however, can increase the likelihood of uncontrolled bleeding. Bypass surgery of coronary arteries, in particular, also carries well known substantial risks. Thus, there is a need for effectively treating cardiovascular disease with fewer of the inherent risks of traditional therapies.

There is also a need for accurate diagnosis of cardiovascular conditions in conjunction with low-risk therapies. A common diagnosis tool is angiography by fluoroscopy. This X-ray technology simply supplies an image of the blood flow within a lumen, thus identifying a stenosis or thrombus, but giving little information about the endovascular wall of a lumen, including its plaque content or other physiological or morphological characteristics. Some important diseases located on non- or minor stenosis regions, such as a vulnerable plaque, can be fatal and are often missed. Furthermore, angiography exposes patients to potentially harmful chemicals and radiation. Other technologies, such as intravascular ultrasound, require expensive additional catheters and potentially dangerous additional procedures that can cause more harm than good and may still not supply sufficient information about the diseased tissue and be beneficial for subsequent treatment. There is currently no option for physicians to gain more optimal information about the lumen wall in an accurate, cost-effective, and efficient manner and provide treatment that presents a reasonable risk profile for the patient.

### SUMMARY OF THE INVENTION

Aspects of the systems and methods of the present invention provide a safe, effective apparatus and method for in vivo characterization and concurrent treatment of diseased body lumen tissue. Embodiments of the invention identify and locate the diseased tissue and the affected surrounding tissue for purposes of diagnosis and subsequent treatment. Embodiments of the invention provide an integrated treatment system that operates in tandem with an identification system.

In an aspect of the invention, an apparatus is provided that includes a catheterized optical probe connected to a spectroscopic analysis system programmed to identify (in vivo) and accurately locate diseased tissue. The catheter further includes an integrated treatment system which, with information provided by the analysis system, can be accurately positioned to effectively treat the diseased tissue such as by application of various therapeutic agents. In an embodiment, the treatment system comprises a needle injection apparatus for injecting various compounds and/or therapeutic agents (e.g. stem cells, antibiotics, gene therapy, neoplasty, etc.) intended for aiding in the treatment of diseased tissue.

In an aspect, an apparatus for probing and treating internal body lumens is provided that includes a catheter having a fiber probe arrangement with one or more treatment lumens and. The system includes an analysis and treatment control system connected to the catheter which is programmed to characterize and locate damaged tissue via the fiber probe arrangement and configured to treat damaged tissue through the one or more treatment lumens.

In an embodiment, the apparatus further comprises a spectrometer connected to said fiber probe arrangement and said treatment control system.

In an embodiment, the apparatus further comprises a needle tip inserter. In an embodiment, the needle tip inserter incorporates a dispersal port for the one or more treatment lumens. In an embodiment, the needle-tip inserter is integrated with the fiber probe arrangement and one or more treatment lumens. In an embodiment, the needle tip inserter is partially retractable within said catheter so as to ease the advancement of said catheter in a patient while permitting optical analysis.

In an embodiment, the analysis and treatment control system is programmed to analyze spectroscopic data, the analysis of the spectroscopic data including distinguishing the types and conditions of tissue within and surrounding a lumen wall. In an embodiment, the spectroscopic data is selected according to predetermined wavelength bands that distinguish levels of particles, gas, and/or liquid contained in the tissue. In an embodiment, the spectroscopic analysis includes the characterization of one or more pathophysiologic or morphologic factors of surrounding tissue within an endovascular region.

In an embodiment, the pathophysiologic or morphologic factors include characterizing the presence, volume, and positioning of plaque within the endovascular region. In an embodiment, the pathophysiologic or morphologic factors further include characteristics of plaque including at least one of collagen content, lipid content, calcium content, inflammation, or the relative positioning of pathophysiologic conditions within the plaque. In an embodiment, characterizing and locating the tissues includes detecting levels of at least one of fibrosis, calcification, or oxygen content. In an embodiment, distinguishing the types and conditions of tissue within and surrounding a patient's lumen includes characterizing and locating tissues associated with at least one of stenosis or thrombosis. In an embodiment, the analysis of said spectroscopic data includes chemometric analysis of said spectroscopic data in relation to previously obtained and stored spectroscopic data. In an embodiment, the chemometric analysis involves at least one technique including Principle Component Analysis (PCA) with Mahalanobis Distance, PCA with K-nearest neighbor, PCA with Euclidean Distance, Partial Least Squares Discrimination Analysis, augmented Residuals, bootstrap error-adjusted single-sample technique, or Soft Independent Modeling of Class Analogy.

In an embodiment, the analysis and control system is configured to perform spectroscopic scans across wavelengths within the range of approximately 300 to 2500 nanometers. In an embodiment, the scans are selectively distributed in sub-ranges of radiation spanning approximately 300 to 1375 nanometers, 1550 to 1850 nanometers, and 2100 to 2500 nanometers.

In an embodiment, the analysis of the spectroscopic data includes estimating relative distances between a distal end of the fiber probe arrangement and tissue analyzed by the spectrometer. In an embodiment, estimating the relative distances includes comparing the magnitudes of spectroscopic absorbance peaks associated with tissue or blood with magnitudes similarly obtained from previously stored spectroscopic absorbance data. In an embodiment, the relative distances includes comparing the magnitudes of the spectroscopic absorbance peaks obtained at different predetermined positions of the catheter relative to the tissue or blood. In an embodiment, estimating the relative distances includes comparing spectroscopic absorbance peaks associated with collection fibers having terminating ends separated longitudinally from each other at a predetermined distance.

In an embodiment, the catheter includes an angle control wire for adjusting the angle of the distal end of the catheter.

In an embodiment, the one or more treatment lumens includes a conduit for delivering a fluid agent to damaged tissue.

In an embodiment, the one or more treatment lumens includes a conduit for delivering therapeutic laser energy.

In an embodiment, the catheter further incorporates one or more sensors. In an embodiment, the one or more sensors includes at least one temperature gauge, pH meter, oxygenation meter, or water content meter.

In an embodiment, the catheter further includes a biopsy sampler.

In an embodiment, the distal end of the catheter includes a guidewire branching from the catheter apart from the needle tip.

In an embodiment, the catheter includes a gripping element about the proximal portion of the catheter, the gripping element having one or more control elements for controlling aspects of positioning the catheter and/or for delivering treatment.

In an aspect, a method for treating body tissue of a lumen is provided including the steps of inserting into a patient a catheter integrated with a fiber optic analysis probe and a treatment delivery conduit, characterizing and locating the lumen tissue to be treated with light delivered and collected through said fiber optic analysis probe, positioning the catheter with information obtained through the fiber optic analysis probe in order deliver treatment to said targeted tissue, delivering a treatment through the treatment delivery conduit.

In an embodiment, the step of delivering treatment through the treatment delivery conduit includes perforating the targeted tissue and injecting a therapeutic agent through the treatment delivery conduit.

In an embodiment, the body lumen treated is a blood vessel. In an embodiment, the blood vessel is a coronary vessel. In an embodiment, the body lumen is a peripheral vessel.

In an embodiment, the tissue of the body lumen treated is associated with at least one of stenosis or thrombosis. In an embodiment, the step of delivering a treatment through the treatment delivery conduit includes the delivery of dipyridamole.

In an embodiment, the lumen treated is an esophagus. In an embodiment, the step of delivering a treatment through the treatment delivery conduit includes the delivery of sclerosant drugs.

In an embodiment, delivering treatment through the treatment delivery conduit includes the injection of therapeutic agents. In an embodiment, the therapeutic agents include at least one of chemical agents, gene therapy agents, stem cell therapy agents, and/or cytotherapy agents. In an embodiment, the therapeutic agents include at least one of heparin, dipyridamole, serine proteinase enzymes and inhibitors, and Apolipoprotein-E, such as for the treatment of stenosis.

In an embodiment, the therapeutic agents include an antibiotic such as for the treatment of an infection. In an embodiment, the therapeutic agents include sclerosant drugs such as for the treatment of dilated vessels (e.g., esophageal varices, varicose veins).

In an embodiment, characterizing and locating the body tissue to be treated includes obtaining spectroscopic data from radiation delivered to and collected from the tissue to be treated via the fiber optic analysis probe and comparing the spectroscopic data with previously stored data characteristic of the type of tissues to be treated in order to identify the type of tissue being analyzed and to locate the position of the tissue being analyzed or treated relative to the catheter.

In an embodiment, characterizing and locating the body tissue includes analyzing spectroscopic data, the analysis of the spectroscopic data including distinguishing the types and conditions of tissue within and surrounding a lumen wall. In an embodiment, the spectroscopic data is selected according to predetermined wavelength bands that distinguish levels of particles, gas, and/or liquid contained in the tissue. In an embodiment, the spectroscopic analysis includes the characterization of one or more pathophysiologic or morphologic factors of surrounding tissue within an endovascular region. In an embodiment, the pathophysiologic or morphologic factors include characterizing the presence, volume, and positioning of plaque within the endovascular region. In an embodiment, the pathophysiologic or morphologic factors further include characteristics of plaque including at least one of collagen content, lipid content, calcium content, inflammation, or the relative positioning of pathophysiologic conditions within the plaque. In an embodiment, characterizing and locating the tissues includes detecting levels of at least one of fibrosis, calcification, oxygen content, lipids, collagen, calcium, hemoglobin, and myoglobin. In an embodiment, distinguishing the types and conditions of tissue within and surrounding a patient's lumen includes characterizing and locating tissues associated with at least one of stenosis or thrombosis. In an embodiment, distinguishing the types and conditions of tissue within and surrounding a patient's lumen includes characterizing and locating tissues associated with at least one of esophageal varices and varicose veins. In an embodiment, the analysis of said spectroscopic data includes chemometric analysis of said spectroscopic data in relation to previously obtained and stored spectroscopic data. In an embodiment, the chemometric analysis involves at least one technique including Principle Component Analysis (PCA) with Mahalanobis Distance, PCA with K-nearest neighbor, PCA with Euclidean Distance, Partial Least Squares Discrimination Analysis, augmented Residuals, bootstrap error-adjusted single-sample technique, or Soft Independent Modeling of Class Analogy.

In an embodiment, obtaining spectroscopic data includes at least one of the methods including diffuse-reflectance spectroscopy, fluorescence spectroscopy, Raman spectroscopy, scattering spectroscopy, optical coherence reflectometery, and optical coherence tomography.

In an embodiment, the spectroscopic data is obtained from radiation spanning wavelengths between approximately 300 to 2500 nanometers. In an embodiment, the spectroscopic data is selectively collected in sub-ranges of radiation spanning approximately 750 to 2500 nanometers, 300 to 1375 nanometers, 1550 to 1850 nanometers, and 2100 to 2500 nanometers.

In an embodiment, the radiation that is delivered and collected through the fiber optic probe is restricted to selectively narrow spans of wavelengths associated with identifying said tissues. In an embodiment, radiation is delivered to tissue or blood within a narrow range including 380 nanometers and scanned across a narrow range including 320 nanometers in order to identify the presence of collagen.

In an embodiment, locating tissues in relation to the catheter includes pre-operative steps of analyzing and comparing the wavelengths and magnitudes of spectroscopic absorbance peaks associated with tissues and blood surrounding the tissues.

In an embodiment, the wavelengths and magnitudes of spectroscopic absorbance peaks associated with tissues and blood is compared with previously obtained and stored spectroscopic absorbance data associated with a catheter approaching similar tissues in a blood medium.

In an embodiment, the distal end of said catheter includes an inserter integrated with the fiber optic probe and delivery conduit, the inserter suitably sharp for perforating targeted tissue.

In an embodiment, during the positioning of the catheter for delivery of treatment, the integrated inserter remains at least partially retracted in the catheter prior to perforation into tissue targeted for treatment and the fiber optic probe is functional while the inserter is at least partially retracted. In an embodiment, final positioning of the catheter for delivery of treatment includes extending the inserter out from the distal end of the catheter into the targeted tissue.

In an embodiment, prior to and during extension of the inserter, a wall of the lumen before which the inserter is positioned is concurrently analyzed and monitored to prevent complete perforation of the inserter through the entire wall of the lumen.

In an embodiment, the prevention of complete perforation includes monitoring the contents of tissue positioned beyond the wall of lumen tissue.

In an embodiment, the therapy agents are chosen and delivered based on data collected during characterizing and locating the body tissue to be treated.

In an embodiment, the release of agents is monitored with the fiber optic probe and controlled using feedback from said monitoring.

In an embodiment, the catheter is introduced into the patient in accordance with a percutaneous transluminal angioplasty.

### BRIEF DESCRIPTION OF THE DRAWINGS

The structure, operation, and methodology of embodiments of the invention, together with other objects and advantages thereof, may best be understood by reading the following detailed description in connection with the drawings in which each part has an assigned numeral or label that identifies it wherever it appears in the various drawings. The drawings are not necessarily to scale, emphasis instead being placed upon illustrating the principles of the embodiments of the invention.

FIG. 1 is a schematic block diagram of an embodiment of an apparatus illustrating the general flow of system control, including identifying, localizing, and treating diseased internal tissues, in accordance with aspects of the invention.

FIG. 2A is an illustrative perspective view of an embodiment of a distal end of a catheterized optical probe and needle injection system that analyzes internal lumens, in accordance with aspects of the invention.

FIG. 2B is a cross-sectional view of the probe and treatment catheter of FIG. 2A taken across lines I-I′.

FIG. 2C is a cross-sectional view of the probe and treatment catheter of FIG. 2A taken across lines II-II′.

FIG. 2D is a cross-sectional view of the probe and treatment catheter of FIG. 2A taken across lines III-III'.

FIG. 2E is a cross-sectional view of the probe and treatment catheter of FIG. 2A taken across lines IV-IV'.

FIG. 3A is an illustrative perspective view of an embodiment of the proximal end of a optical probe and needle injection catheter that analyzes internal lumens, in accordance with aspects of the invention.

FIG. 3B is a cross-sectional view of the probe and treatment catheter of FIG. 3A taken across lines I-I'.

FIG. 3C is a cross-sectional view of the probe and treatment catheter of FIG. 3A taken across lines II-II'.

FIG. 4A is a schematic cross sectional view of an embodiment of a probe and treatment catheter within a body lumen across its longitudinal axis according to aspects of the invention.

FIG. 4B is a schematic cross sectional view of an embodiment of a probe and treatment catheter within a body lumen along its longitudinal axis according to aspects of the invention.

FIG. 5A is an illustrative cross-sectional view of an embodiment of the distal end of a catheter deployed in a body lumen according to aspects of the invention.

FIG. 5B is an illustrative cross-sectional view of an embodiment of the deployed catheter of FIG. 5A with a needle tip inserter engaged with adjacent tissue according to aspects of the invention.

FIG. 6A is an illustrative perspective view of an embodiment of the distal end of a probe and treatment catheter according to another aspect of the invention.

FIG. 6B is a cross-sectional view of the probe and treatment catheter of FIG. 6A taken across lines I-I'.

FIG. 6C is a cross-sectional view of the probe and treatment catheter of FIG. 6A taken across lines II-II'.

FIG. 6D is a cross-sectional view of the probe and treatment catheter of FIG. 6A taken across lines III-III'.

FIG. 6E is a cross-sectional view of the probe and treatment catheter of FIG. 6A taken across lines IV-IV'.

FIG. 7A is an illustrative cross-sectional view of an embodiment of the distal end of a catheter deployed in a body lumen according to aspects of the invention.

FIG. 7B is an illustrative cross-sectional view of an embodiment of the deployed catheter of FIG. 7A with a needle tip inserter engaged with adjacent tissue according to aspects of the invention.

### DETAILED DESCRIPTION OF EMBODIMENTS

The accompanying drawings are described below, in which example embodiments in accordance with the present invention are shown. Specific structural and functional details disclosed herein are merely representative. This invention may be embodied in many alternate forms and should not be construed as limited to example embodiments set forth herein.

Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on,” “connected to” “abutting,” “coupled to,” or “extending from” another element, it can be directly on, connected to, abutting, or coupled to the other element or intervening elements may be present. In contrast, when an element is referred to as being “directly on,” “directly connected to,” “directly abutting,” “directly coupled to,” or “directly extending from” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

In order to overcome the limitations described above, an apparatus and method are provided for treating tissue surrounding a lumen by integrating an inspection system for locating tissue to be treated with a treatment delivery system.

Aspects of the invention employ spectroscopic analysis with any two or more single wavelengths or one or more narrow wavelength bands, or a whole wavelength range to identify and localize diseased lumen tissue in vivo. The light signal scattered or emitted from an illuminated area provides information about a change in tissue chemical components (such as water content, oxygenation, pH value, collagen, proteoglycans, calcium), tissue structures (such as cell size, types), inflammatory cellular components (such as T lymphocytes, macrophages, and other while blood cells), that help characterize states of diseased or damaged tissue.

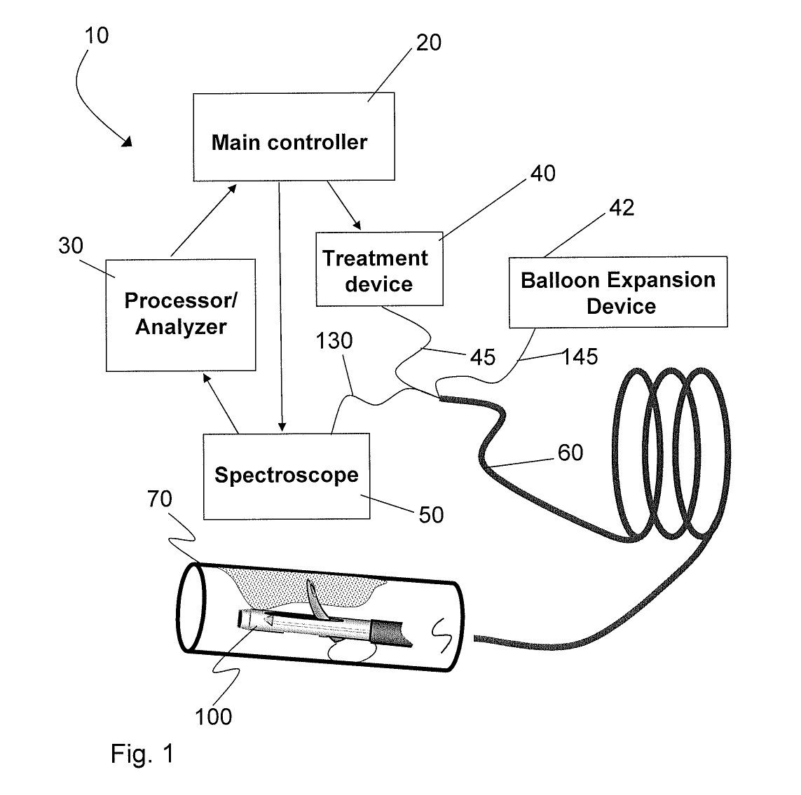

FIG. 1 is a schematic block diagram of a system 10 illustrating an embodiment of the general flow of control, including identifying, localizing, and treating diseased internal tissues, in accordance with aspects of the invention. A main controller 20 coordinates operation of a treatment delivery device 40, a spectroscope 50, a processor/analyzer 30, a balloon expansion device 42, and a treatment delivery probe 100 shown with its distal end deployed within a lumen 70. A signal initiated by an operator (e.g., through a manipulable/graphical user interface) can instruct main controller to perform analysis of a lumen wall 70 via the spectroscope 50, which has fibers 130 connected through cabling 60 to the treatment delivery probe 100. Signals collected by the spectroscope 50 can be further processed (e.g., by chemometric analysis) by processor/analyzer 30 after which the processed data (e.g., identified tissue types/locations) can be transmitted to the main controller 20. Data collected and processed can be stored, displayed, and/or transmitted via main controller 20 for use by an operator and/or used for automated operation of treatment device 40 and/or balloon expansion device 42. A flush lumen 145 provides a conduit through which balloon media can be delivered/removed by balloon expansion device 42. An operator and/or main controller 20 can signal an anchoring balloon to fix the position of the catheter and signal treatment device 40 to deliver controlled types and amounts of treatment therapy to lumen 70.

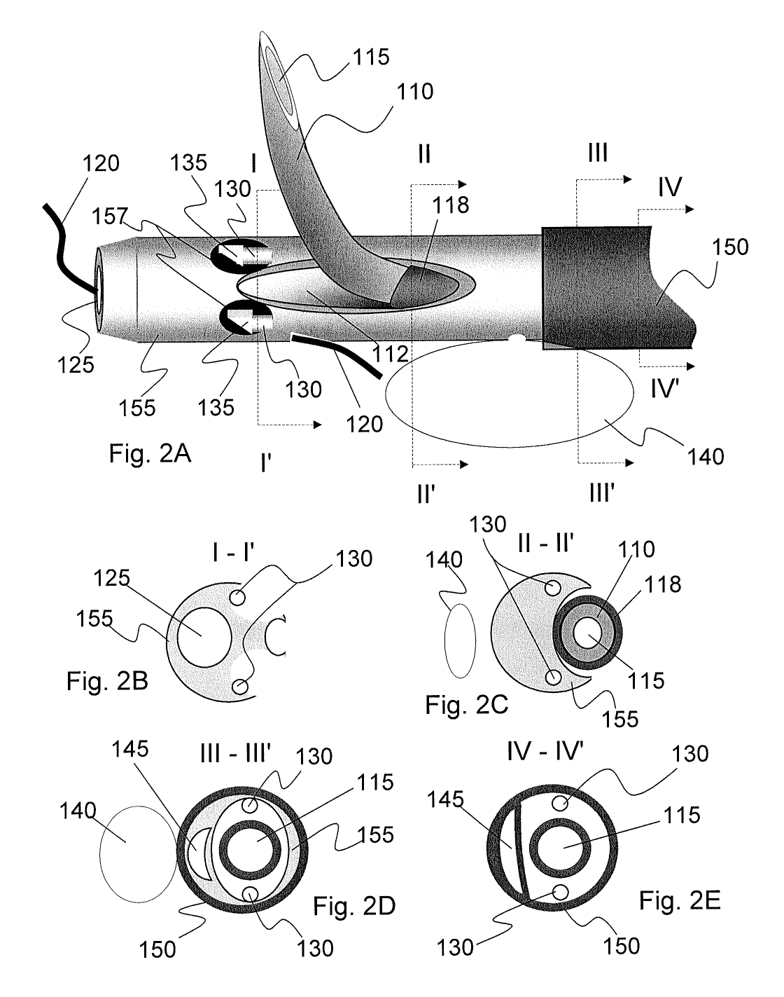

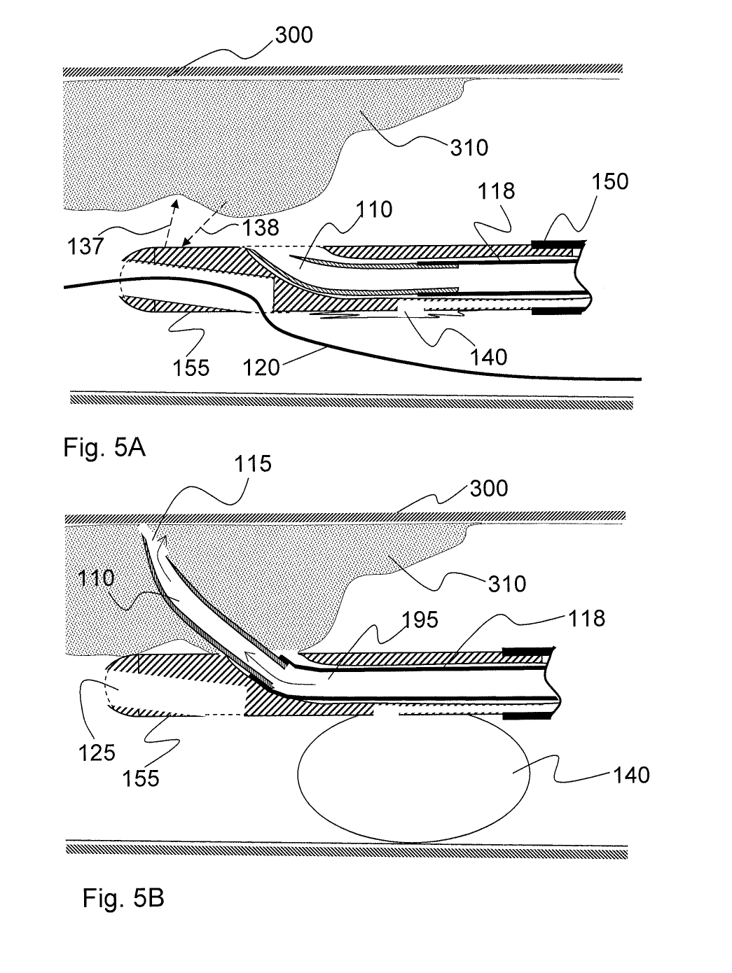

FIG. 2A is an illustrative perspective view of an embodiment of a distal end of a catheterized optical probe and needle injection system that analyzes internal lumens, in accordance with aspects of the invention. FIG. 2B is a cross-sectional view of the probe and treatment catheter of FIG. 2A taken across lines I-I'. FIG. 2C is a cross-sectional view of the probe and treatment catheter of FIG. 2A taken across lines FIG. 2D is a cross-sectional view of the probe and treatment catheter of FIG. 2A taken across lines FIG. 2E is a cross-sectional view of the probe and treatment catheter of FIG. 2A taken across lines IV-IV'. In an embodiment, a distal end of a probe and needle injection catheter includes a protective catheter sheath 150 within which is a catheter head 155 at its distal end. Extending through catheter sheath 150 are at least two fibers 130, one designated for delivery of radiation to adjacent tissue and another designated for collection of radiation from adjacent tissue. The fibers can be configured as a fiber probe arrangement. Each fiber 130 has an associated redirecting component 135 for directing light between fibers 130 and adjacent tissue. These redirecting components can include, for example, side-firing fiber ends (i.e., beveled tips with reflective coated ends), mirrors, prisms, and/or lenses or etched fibers such as those described in co-pending and commonly owned U.S. patent application Ser. No. 12/466,503, filed on May 15, 2009, entitled “SHAPED FIBER ENDS AND METHODS OF MAKING SAME”, the entire contents of which is herein incorporated by reference. Openings 157 are positioned along catheter head 155 so as to allow the travel of light between the outside of the catheter and redirecting components 135. In an embodiment, openings 157 can be covered with a translucent material (e.g., plastic, glass) so as to better protect the redirecting components 135 and fibers 130. A needle injection component of the catheter includes a treatment delivery tube 118 which runs from the proximal end of the catheter to a connecting point with an inserter 110 which can slidably engage or at least partially retract through an opening 112 in catheter head 155. The inserter 110 can be integrated with a fiber probe arrangement. Inserter 110 preferably comprises stainless steel or similar material suitable for perforating, penetrating, or piercing adjacent tissue by moderate forward pressure. In an embodiment, the needle has a gauge (size) of between about 23-31 (i.e., an outer diameter of between about 0.3 and 0.8 mm). The treatment delivery tube 118 and inserter 110 provide a conduit 115 through which treatment agents may be delivered to adjacent tissue. A guidewire conduit 125 allows for a guidewire 120 to be used for deployment of the catheter. In order to secure and anchor the position of the catheter for local treatment delivery, an anchoring balloon 140 can be inflated as to appose the distal end of the catheter directly in place with surrounding tissue. While anchored in place for the delivery for treatment, needle inserter 110 may be engaged with adjacent tissue 310 (e.g., as shown in FIG. 5B). The anchoring balloon 140 allows for the distal end of the catheter to remain relatively stationary with respect to adjacent tissue.

In an embodiment, an apparatus such as contemplated by FIGS. 2A-2E is employed in body lumens generally greater than about 2 millimeters (e.g., esophagus and peripheral blood vessels). In an embodiment, treatment is applied for such conditions as blood dilated lumens including, for example, esophageal varices, varicose veins, etc., such as with sclerosant drugs. In an embodiment, an apparatus employs gene therapy agents, stem cell therapy agents, cytotherapy agents, heparin, dipyridamole, serine proteinase enzymes and inhibitors, and/or Apolipoprotein-E. Various such therapies can be useful for the treatment of cancer, for the regeneration of dead or highly damaged tissue, and/or cardiovascular diseases including atherosclerosis and others that result in stenosis and/or thrombosis (blood clots). Effective localized dosages of dipyridamole for the treatment and prevention of stenosis and thrombosis, for example, is described in U.S. Patent Application No. 60/867,438 filed Nov. 28, 2006, and International Patent Cooperation Treaty Application No. PCT/U.S.07/85570 filed on Nov. 27, 2007, the entire contents of each of which is herein incorporated by reference. The application of various gene therapies also provide promising results for the reduction of plaque in a body lumen. In an embodiment, the therapeutic agents include an antibiotic such as for the treatment of an infection. In many instances, precise, local delivery of such agents can improve treatment and substantially avoid the side-effects and risks involved with more general and/or systemic delivery. The catheter may also provide a conduit through which other treatment tools can deliver treatment to the affected area, e.g. additional treatment lumens or a treatment fiber with high power laser energy to canalize infarct tissue for revascularization as described by Lauer B., et al., “Catheter-based percutaneous myocardial laser revascularization in patients with end-stage coronary artery disease.” J Am Coll Cardiol. 1999 Nov. 15; 34(6):1663-70, incorporated herein in its entirety by reference. For example, in embodiments of the invention, one or more of fibers 130 could be adapted and used to deliver therapeutic laser energy. These fibers could be, for example, switched between use for delivery/collection for purposes of analysis and use for delivering therapeutic laser energy. In other embodiments, the inventive catheter incorporates a biological, electric, or chemistry-based sensor or tool connected with a metal fiber, or other structural or reinforcing wire elements permitting additional diagnosis or monitoring of target tissue, e.g. tissue temperature, pH, oxygenation, water content, other chemical composition and/or even tissue biopsy via the catheter head. In an embodiment, the catheter includes one or more sensors. The sensors can be at least one of a temperature gauge, pH meter, oxygenation meter, and water content meter. In another embodiment, the catheter includes a biopsy sampler. In an embodiment, a sensor wire can travel along a similar path as that of fibers 130 and a sensor/transducer could be situated in, for example, needle inserter 110. In an embodiment, a biopsy can be performed by extracting tissue or other materials through needle inserter 110 and suctioning them to the proximal end of the catheter. A cutting device (not shown) could be incorporated into needle inserter 110 in order to detach tissue for extraction.

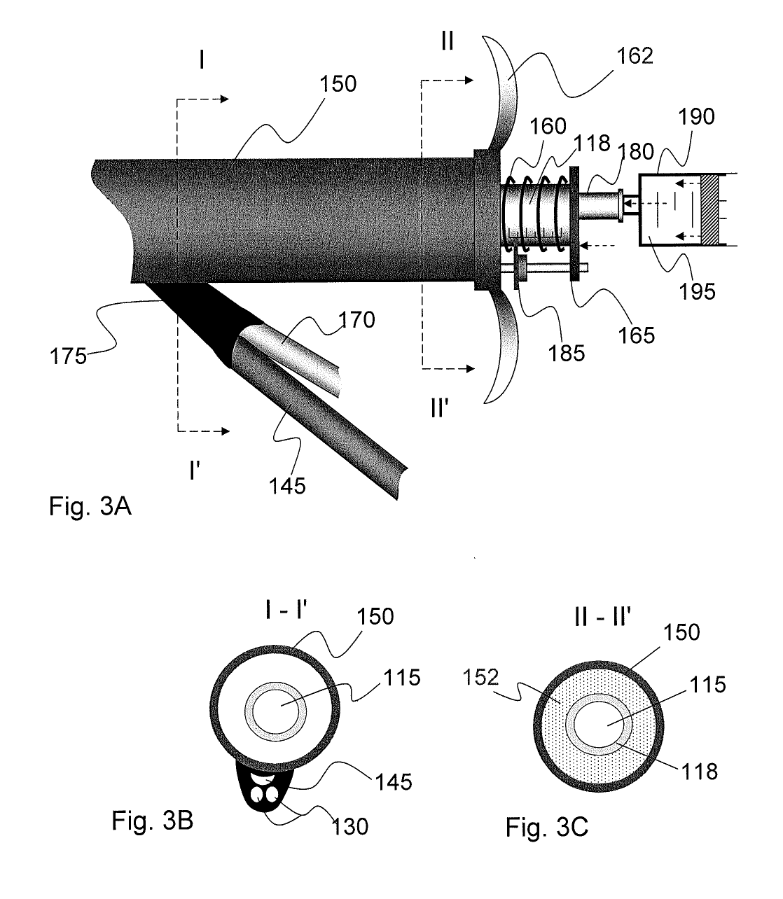

FIG. 3A is an illustrative perspective view of an embodiment of a proximal end of an optical probe and needle injection catheter that analyzes internal lumens, in accordance with aspects of the invention. FIG. 3B is a cross-sectional view of the probe and treatment catheter of FIG. 3A taken across lines I-I'. FIG. 3C is a cross-sectional view of the probe and treatment catheter of FIG. 3A taken across lines II-II′. In an embodiment, the proximal end of a catheter probe and needle injection system includes a protective catheter sheath 150. An interface for therapy delivery protrudes from the end of sheath 150 that includes a needle insertion push lever 165 that can slidably move treatment delivery tube 118 so that the inserter 110 is driven out of catheter head 155 (see FIGS. 2A-2E). Needle insertion push lever 165 is spring loaded by a spring 160 so that inserter 110 is automatically at least partially retracted upon release of lever 165. A gripping element 162 allows an operator to stabilize the proximate end of the catheter while engaging lever 165. An indicator 185 provides a guide as to the amount inserter 110 protrudes from catheter head 155. For example, if information provided by the optional probe system described herein indicates an optimal position for therapy to be delivered from the catheter head, the lever 165 can be correspondingly engaged to place the needle in the desired position. The mechanism promotes a reduced risk of puncturing the walls of the lumen within which the catheter is deployed and can allow for more optimal placement of therapeutic delivery. A therapy delivery interface 180 provides an opening for therapeutic agents to be delivered through conduit 115 to the needle inserter 110. In an embodiment, a syringe 190 operates to administer a treatment agent 195 through the delivery interface 180. Other mechanical means (e.g., automated pumps, etc.) can also be used to administer therapy.

Leading up to a junction 175 with catheter sheath 150, an insulating conduit 170 encloses and protects fibers designated for the delivery and/or collection of radiation to/from adjacent tissue, for example, fibers 130 shown in FIGS. 2A-2E. A flushing lumen 145 also connects with, and intersects, catheter sheath 150 at junction 175. A solid ring 152 is positioned within sheath 150 so as to block the backflow of blood through the proximal end of the catheter. The solid ring 152 can be formed of materials such as rubber or plastic.

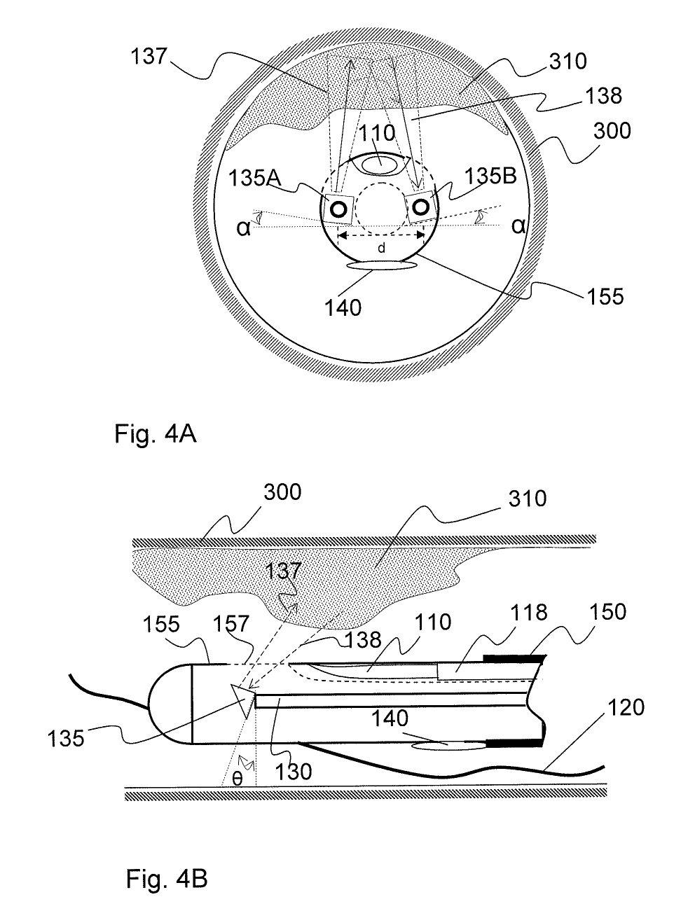

FIG. 4A is a schematic cross sectional view of an embodiment of a probe and treatment catheter within a body lumen across its longitudinal axis according to aspects of the invention. FIG. 4B is a schematic cross sectional view of a probe and treatment catheter within a body lumen along its longitudinal axis according to an embodiment. Incident light (represented by exemplary transmission paths 137) is directed from a delivery fiber tip 135A toward targeted tissue 310 which can be, for example, diseased tissue along the lumen wall 300. After incident light interacts with tissue 310, light is directed from targeted tissue 310 toward a collection fiber tip 135B (such as along exemplary transmission paths 138). As described in accordance with methods described above, the collected light can then be processed in order to provide diagnostic and positional information about targeted tissue 310. Processed information can then be used to position the catheter and determine the amount and type of treatment agent to be delivered with treatment delivery tube 118 and inserter 110. In an embodiment, the separation distance d between fiber tips 135A and 135B and the angles α and θ of the tips are predetermined to aid in the calculation of the depth and location of radiation delivered and/or collected by the tips. For example, the probe separation distance between delivery/collection output/input and the angles of emission and/or collection of radiation can be used in a manner known to those of ordinary skill in the art to help determine the location and depth of targeted tissue associated with the type and magnitude of received signals. The amount of detectable signal and the depth of the path of the collected signal is generally proportional to the degree of separation between delivery and collection fibers. While having signal power levels sufficiently low not to damage targeted tissue, a separation of less than about 1.5 mm is preferable for receiving an adequate collection signal. In various embodiments, tips 135A and 135B can be shaped fiber ends such as “side-fire” tips cleaved at predetermined angles with reflective coatings for directing radiation along predetermined paths between fibers 130 and targeted tissue. In an embodiment, tips 135A and 135B comprise light redirecting elements including lenses, mirrors, prisms, and the like. Examples of various embodiments of light redirecting arrangements are more fully described in, for example, co-pending U.S. patent application Ser. No. 11/537,258 filed on Sep. 29, 2006, published as U.S. Patent Application Publication No. US20070078500 A1, U.S. patent application Ser. No. 11/834,096 filed Aug. 6, 2007, published as U.S. Patent Application Publication No. US20070270717 A1, and U.S. Patent Application No. 61/025,514 filed on Feb. 1, 2008, and U.S. patent application Ser. No. 11/762,956 filed Jun. 14, 2007, the entire contents of each of which is herein incorporated by reference.

FIG. 5A is an illustrative cross-sectional view of an embodiment of the distal end of a catheter deployed in a body lumen according to aspects of the invention. FIG. 5B is an illustrative cross-sectional view of the deployed catheter of FIG. 5A with a needle tip inserter engaged with adjacent tissue according to an embodiment. While the catheter is placed within a lumen, analysis can be performed through the delivery of signals 137 and collection of signals 138 in order to locate and diagnose tissue for potential treatment such as described herein above. Once targeted tissue has been diagnosed and located with the catheter, the catheter can be positioned (if necessary) for the delivery of treatment agents through needle inserter 110. In order to secure and anchor the position of the catheter for local treatment delivery, balloon 140 can then be inflated (such as shown in FIG. 5B) so as to appose the distal end of the catheter directly in place with surrounding tissue. While anchored in place for the delivery for treatment, needle inserter 110 may be engaged with adjacent tissue 310 (as shown in FIG. 5B) through a mechanism for slidably translating delivery tube 118 and inserter 110 into adjacent tissue such as described further above (e.g., see FIG. 3A and accompanying description). Once engaged into adjacent tissue, predetermined levels of treatment agent 195 can be delivered through the catheter, exiting from the tip of inserter 110 at port 115 and into the targeted tissue 310.

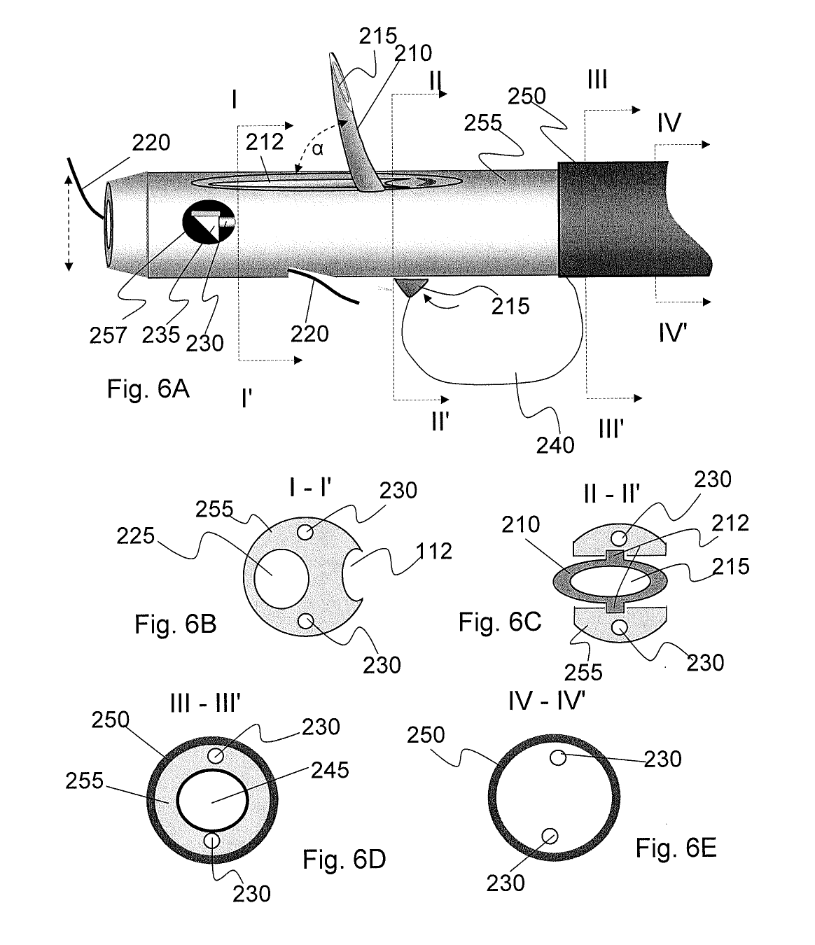

FIG. 6A is an illustrative perspective view of the distal end of a probe and treatment catheter according to another embodiment of the invention. FIG. 6B is a cross-sectional view of the probe and treatment catheter of FIG. 6A taken across lines I-I'. FIG. 6C is a cross-sectional view of the probe and treatment catheter of FIG. 6A taken across lines FIG. 6D is a cross-sectional view of the probe and treatment catheter of FIG. 6A taken across lines FIG. 6E is a cross-sectional view of the probe and treatment catheter of FIG. 6A taken across lines IV-IV'. In an embodiment, treatment agents are supplied through a lumen 245 that both inflates an anchoring balloon 240 and administers the treatment agent through a connected lumen 215 within a needle inserter 210. As treatment agent is supplied, the subsequent inflation of balloon 240 causes needle inserter 210 to rotate about pivot points 230 and out through an opening 212 at an angle α relative to a direction of extension of the catheter and cause the needle inserter 210 to penetrate adjacent tissue and deliver treatment thereto. In an embodiment, angle α is at least about 45 degrees. The catheter includes a catheter sheath 250 within which is a catheter head 255 that partially protrudes from the distal end of sheath 250. In accordance with various embodiments described herein, at least two optical fibers 230 extend to openings 257 which allow for light to be delivered and/or collected through terminating ends 235. A guidewire conduit 225 allows for a guidewire 220 to be used for deployment of the catheter. In an embodiment, a catheter assembly in accordance with the invention can be cost-effectively manufactured for very small sized lumens (e.g., coronary arteries of less than about 2 to 3 millimeters) and have maximum outer diameters as small as about 1.5 mm or less. In an embodiment, the needle inserter 210 has a gauge (size) of between about 23-31 (i.e., an outer diameter of between about 0.3 and 0.8 mm).

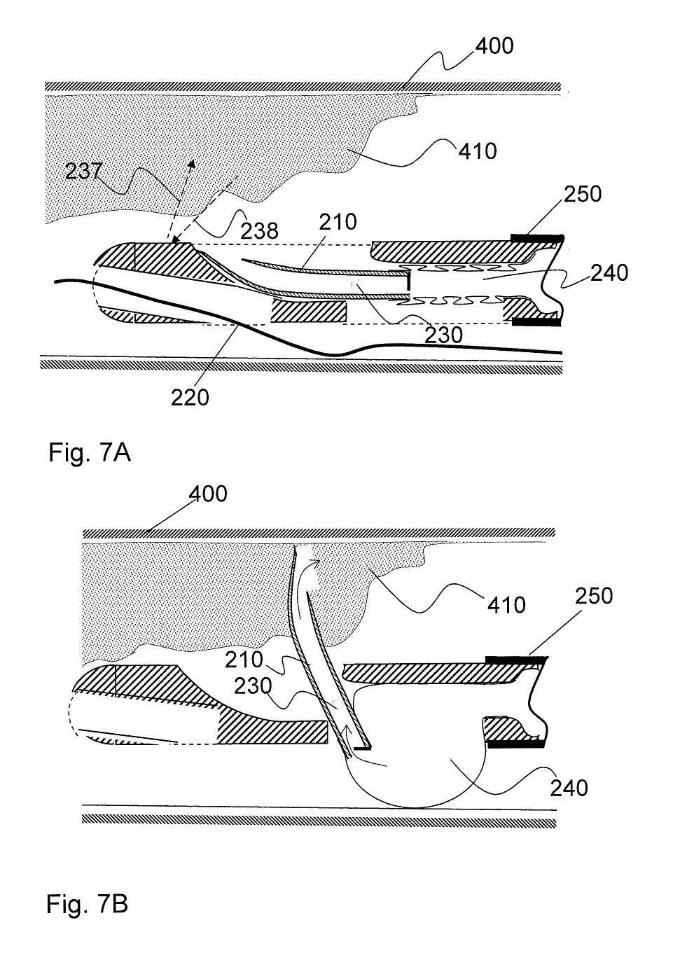

FIG. 7A is an illustrative cross-sectional view of an embodiment of the distal end of a catheter deployed in a body lumen 400 according to aspects of the invention. FIG. 7B is an illustrative cross-sectional view of the deployed catheter of FIG. 7A with a needle tip inserter engaged with adjacent tissue 410 according to aspects of the invention. While the catheter is placed within a lumen (e.g., such as in accordance with percutaneous transluminal angioplasty) analysis can be performed through the transmission of light (e.g., along path 237) and collection of return signals (e.g., along path 238) in order to locate and diagnose tissue for potential treatment such as described herein above. Once targeted tissue has been diagnosed and located with the catheter, the catheter can be positioned (if necessary) for the delivery of treatment agents through needle inserter 210. In order to secure and anchor the position of the catheter for local treatment delivery, balloon 240 is inflated (such as shown in FIG. 7B) so as to fix the distal end of the catheter directly in place with respect to adjacent tissue while simultaneously rotating needle inserter 210 about axis 230 and causing the needle to engage adjacent tissue. Balloon 240 is pressurized with treatment agent that also exits in a controlled manner from the tip of inserter 210 and into adjacent tissue 410. In an embodiment, supply of the treatment/balloon inflation media is protected with a pressure release system (such as those known to one of ordinary skill in the art) so as to prevent over-pressurization that could cause a inadvertent puncture of the lumen wall by the injection needle 210.

In an embodiment, the probe aspect of a system such as described herein monitors the progress of a needle inserter as it engages in adjacent tissue and/or the progress of delivering treatment agent. Data collected from the probe system can help determine the optimal depth of the needle for treatment delivery and/or prevent an inadvertent complete perforation, penetration, or piercing of a vessel wall. In an embodiment, the supply (e.g., pressure) of treatment agent and/or balloon inflation media delivered is coordinated with monitoring of the inserter's progress such that the inserter may be stopped if an indication of imminent perforation is measured.

### Spectroscopic Tissue Analysis and Diagnosis

A number of techniques with the use of embodiments of the invention, including spectroscopy, can be employed for diagnosing tissue conditions. Spectroscopic analysis techniques used alone or in combination include, but are not limited to, fluorescence spectroscopy, visible spectroscopy, diffuse-reflectance spectroscopy, infrared or near-infrared spectroscopy, scattering spectroscopy, optical coherence reflectometery, optical coherence tomography, and Raman spectroscopy.

To optimize speed, it is preferable that, during operation, the source of radiation be limited and selectable in particular wavelength band ranges known to provide optimal feedback about the types of tissue being targeted (e.g. diseased/damaged tissues within and surrounding vessels). A variety of light sources can be used to provide radiation in this manner, such as one or multiple lasers, one or multiple LEDs, a tunable laser with one or multiple different wavelength ranges, Raman amplifier lasers, and a high-intensity arc lamps. These light sources can provide the desired optical radiation region by sequential tunable scanning or by simultaneously spanning the desired wavelength band(s). Wavelength tuning during scans should preferably occur between about a couple of microseconds to less than one second in order to avoid motion related artifacts (e.g. those associated with a pulsing heart).

In embodiments of the invention, data from multiple similar spectra scans across varying wavelength ranges with known varying backgrounds in multiple living or deceased subjects can be compiled and analyzed to develop a model to be programmed in coordination with optical, processor/analyzer, and controller components of embodiments of the invention described herein

Referring back to FIG. 1, in an embodiment, a detector and processor/analyzer (such as, for example, the spectroscope 60 and processor/analyzer 30 perform spectroscopic scans across wavelengths having a range of approximately 300-2500 nm. In an embodiment, the spectroscopic absorbance data is collected across sub-ranges of radiation spanning approximately 300-1375 nm., 1550-1850 nm., and 2100-2500 nm. In an embodiment, radiation is delivered to tissue or blood at a narrow range including 380 nanometers and scanned across a narrow range including 320 nanometers in order to identify the presence of collagen. Examples of techniques for measuring the presence of blood are described in, for example, U.S. Patent Application No. 60/945,481 filed Jun. 21, 2007, the entire contents of which is herein incorporated by reference.

Additional optical elements may be integrated into the delivery and collection systems in order to improve the quality of and/or provide additional control over signals. For instance, filters of various types (e.g. longpass, lowpass, bandpass, polarizing, beam splitting, tunable wavelength, etc.) could be placed in between the light source and delivery fibers or between the detector and collection fibers depending on application parameters. For example, a coating of appropriate polymer or glass on the ends of fibers could serve as a filter.

A number of different types of detectors may be suitable for initial collection and signal processing of radiation received through collection fibers. A detection device may include one or more (individual or arrayed) detector elements at the proximal portion of collection fiber(s) in accordance with embodiments of the invention, such as InGaAs, Silicon, Ge, GaAs, and/or lead sulfide detectors for detecting optical radiation emitted from illuminated tissue.

The detector converts the collected optical signal into an electrical signal, which can be subsequently processed into spectral absorbance or other data using various known signal processing techniques. The electrical signal is preferably converted to digital spectral data for further processing using one or more discrimination algorithms. Using collected spectral data, discrimination algorithms may execute morphemetry measurements, chemical analysis, or perform similar calculations and correlate the results with pre-stored model data to provide a diagnosis of targeted tissue. Model data representing the relationship between spectral data and tissue characteristics is preferably developed from the analysis of large amounts of patient in vivo data or ex vivo data simulating in vivo conditions. The models can be developed with chemometric techniques such as Principle Component Analysis (PCA) with Mahalanobis Distance, PCA with K-nearest neighbor, PCA with Euclidean Distance, Partial Least Squares Discrimination Analysis (PLS-DA), augmented Residuals (PCA/MDR), and others such as the bootstrap error-adjusted single-sample technique (BEST), and Soft Independent Modeling of Class Analogy (SIMCA).

For aiding in a careful approach and interrogation (e.g. preventing perforation of a vessel wall into an outside fat layer) by the inventive probe, absorbance peaks for distinguishing the myocardium, fat, blood, collagen and/or fibrin are discernable with use of the above described algorithmic techniques. Several high-speed commercially available near infrared spectrometers are available for obtaining the desired spectral readings including the IntegraSpec™ NIR Microspectrometer from Axsun Technologies, Inc., the Antaris FT-NIR spectrometer, and a FOSS NIR System, model 6500. The models were selected for their high speed and performance in the spectral regions of interest (i.e. near infrared). A number of other comparable high-speed spectrometers would also be suitable. Limiting scanning to generally flat, narrow regions of spectroscopic bands (e.g. 1550 to 1800 nanometers) is preferable for purposes of speed while maintaining reasonable accuracy. In an embodiment, spectroscopic scans are performed across wavelengths having a range of approximately 300-2500 nm. While probing for particular tissue/fluid types or conditions, it may be preferable to employ such techniques as tissue fluorescence spectroscopy and/or selectively focus transmission bands to excite specific scanning ranges. For example, a radiation excitation peak for collagen at approximately 380 nm occurs when radiation of approximately 340 nm is delivered.

In order to accurately position the catheter for providing treatment, spectroscopic analysis can also distinguish the types and conditions of tissue within and surrounding the target lumen. The chosen discrimination algorithm can compare collected data with pre-programmed spectra data of diseased tissue to categorize both the condition and relative location (to the catheter tip) of a tissue area. Based on spectral analysis, the tissue can be characterized as being diseased, normal, or otherwise affected tissue within or surrounding the lumen.

The intensity of peaks associated with various tissue types can generally be correlated with the distance the probe is from the targeted tissue and from data related to the medium in which the probe is in (e.g. collagen, blood, vessel wall tissue, fat). Thus, analysis of spectroscopic absorbance data can include estimating relative distances between a distal end of a fiber probe arrangement and tissue to be analyzed. For instance, in preparing and programming an embodiment for operation, experiments can be performed on various in vivo or ex vivo samples, including samples having measured thicknesses of layers of these types of tissues. Fat tissue surrounding a vessel is known to generate particular absorbance peaks. Data can be collected on the changes (e.g. intensity) in these peaks as the needle tip of an embodiment approaches the tissue during deployment. Collected data would correlate, for example, peak intensity with the otherwise measured distances between the needle tip and the vessel wall so as to help avoid inadvertent puncture.

In another example of pre-operational model data gathering, a probe in accordance with an embodiment could be placed in a blood medium at the appropriate temperature (i.e. 38° C.) with its position modified relative to targeted tissue (e.g. vessel wall tissue, fat tissue). The tissue types and their positions in relation to the probe would be known independently of data gathered from the probe to develop additional chemometric correlation models. This analysis would be useful for positioning and entry into the vessel wall by needle tip during actual operation.

Embodiments also provide for enhanced tracking (real-time) the position of the distal end of the catheter as analysis is performed, providing enhanced calculations of the size, shape, and/or development of a diseased/damaged area and transitions of tissue conditions therein. This information is highly useful for assessing the best area for applying treatment such as, for example, the affected areas surrounding an area of diseased, damaged, and/or necrotic tissue. A number of technologies are commercially available for enhanced real-time tracking of catheter movement, including, for example, fluoroscopy-based solutions, magnetic resonance imaging (MRI), image-guidance, rotary and linear translation, and precision encoders. Embodiments of the invention include features and materials (e.g. radiopaque materials) within the distal end of catheters detectable by, for example, a fluoroscope or MRI. For example, needle inserters 110 and 210 of catheter bodies 155 and 255 (as shown, respectively, in FIGS. 2A-2E and FIGS. 6A-6E) can include a highly radiopaque material such as, for example, platinum or gold detectable by a fluoroscope. In an embodiment, a controller (e.g. controller 20 of FIG. 1) can receive data from a tracking device (e.g. a fluoroscope) while the processor/analyzer receives simultaneously collected data from the probe end of the catheter so as to track and calculate the geometry, size, and position of targeted tissue within a patient.

In an embodiment, a computer-aided output, such as visual representation, e.g. a graph or other output, or an audible presentation, can be provided to indicate to the operator the characterization of the diseased/damaged tissue, including whether the tissue area falls within one or more categories described above and/or to display the relative position of a suitable treatment area. The algorithms described above can be programmed into a central system processor and/or programmed or embedded into a separate processing device, depending on speed, cost, and other practical considerations.

Embodiments can also be adapted for studying the development of diseased tissues and assessing the effectiveness of treatment. After treatments are applied with use of the invention, for instance, the inventive catheter can be reinserted to assess the development and progress of the targeted areas. Information about the treatments and assessed tissue conditions can be recorded within the inventive system for purposes of determining future treatments and for conducting studies to optimize treatment plans in other patients.

It will be understood by those with knowledge in related fields that uses of alternate or varied forms or materials and modifications to the apparatus and methods disclosed are apparent. This disclosure is intended to cover these and other variations, uses, or other departures from the specific embodiments as come within the art to which the invention pertains.

What is claimed is:

1. An apparatus for probing and treating internal body organs comprising:
a catheter having a fiber probe arrangement and one or more treatment lumens;
a needle-tip inserter integrated with said fiber probe arrangement and said one or more treatment lumens;
a spectrometer connected to said fiber probe arrangement;
an analysis and treatment control system connected to said catheter and spectrometer, said analysis and control system programmed to characterize and locate damaged tissue within a wall of a body lumen and to treat said damaged tissue with said needle-tip inserter and said one or more treatment lumens.

2. The apparatus of claim 1, wherein said needle tip inserter comprises the probe ends of one or more fibers of said fiber probe arrangement and a fluid dispersal port for said one or more treatment lumens.

3. The apparatus of claim 1, wherein said needle tip inserter is at least partially retractable within said catheter so as to ease the advancement of said catheter in said internal body organs while permitting optical analysis.

4. The apparatus of claim 1, wherein the analysis and treatment control system is programmed to analyze spectroscopic data, the analysis of the spectroscopic data including distinguishing the types and conditions of tissue within and surrounding the wall of the body lumen.

5. The apparatus of claim 4, wherein distinguishing the types and conditions of tissue within and surrounding the wall of a body lumen includes characterizing and locating tissues associated with at least one of stenosis or thrombosis.

6. The apparatus of claim 5, wherein characterizing and locating the tissues associated with stenosis or thrombosis includes detecting levels of at least one of collagen content, lipid content, inflammation, fibrosis, calcification, oxygen content, and the relative positioning of pathophysiologic conditions within the plaque.

7. The apparatus of claim 1, wherein said analysis and control system is configured to perform spectroscopic scans across wavelengths in the range of approximately 300 to 2500 nanometers.

8. The apparatus of claim 1, wherein the analysis and control system is configured to estimate relative distances between the distal end of said fiber probe arrangement and tissue analyzed by said spectrometry comparing spectroscopic absorbance peaks associated with collection fibers of said fiber probe arrangement having terminating ends separated from each other at a predetermined distance.

9. The catheter of claim 1 further comprising an angle control wire for adjusting the angle of the distal end of said catheter.

10. A method for treating a body lumen, said method comprising:
inserting into a patient's lumen a catheter integrated with a fiber optic analysis probe, an integrated inserter for perforating targeted tissue, and a treatment delivery conduit;
characterizing and locating the tissue of the lumen to be treated with radiation delivered and collected through said fiber optic analysis probe;
positioning said inserter to perforate targeted tissue and deliver treatment with information obtained through said fiber optic analysis probe; and
injecting a therapeutic agent through said treatment delivery conduit.

11. The method of claim 10, wherein the body lumen to be treated is associated with at least one of stenosis or thrombosis.

12. The method of claim 10, wherein characterizing and locating tissue of the lumen to be treated comprises:
obtaining spectroscopic data from radiation delivered to and collected from said tissue to be treated via said fiber optic analysis probe; and
comparing said spectroscopic data with previously stored data characteristic of a lumen in order to identify the type of tissue being analyzed and to locate the position of said tissue being analyzed relative to said catheter.

13. The method of claim 12, wherein obtaining spectroscopic data comprises at least one of the methods including diffuse-reflectance spectroscopy, fluorescence spectroscopy, Raman spectroscopy, scattering spectroscopy, optical coherence reflectometery, and optical coherence tomography.

14. The method of claim 10, wherein at least one of gases, fluids, and compounds are analyzed, said at least one of gases, fluids, and compounds are selected from the group including collagen, calcium, oxygen, hemoglobin, and myoglobin.

15. The method of claim 10, wherein characterizing the tissue to be treated involves chemometric analysis selected from the group of techniques consisting of Principle Component Analysis (PCA) with Mahalanobis Distance, PCA with K-nearest neighbor, PCA with Euclidean Distance, Partial Least Squares Discrimination Analysis, augmented Residuals, bootstrap error-adjusted single-sample technique, and Soft Independent Modeling of Class Analogy.

16. The method of claim 10, wherein the spectroscopic data is obtained from radiation spanning wavelengths between approximately 750 to 2500 nanometers.

17. The method of claim 16, wherein radiation is delivered to tissue or blood at a wavelength of about 380 nanometers and scanned to detect fluorescence at about 320 nanometers in order to identify the presence of collagen.

18. The method of claim 10, wherein, during positioning of said catheter for delivery of treatment, said integrated inserter for perforating targeted tissue remains at least partially retracted in said catheter prior to perforation into tissue targeted for treatment.

19. The method of claim 10, wherein, prior to and during extension of said inserter, a wall of the lumen before which said inserter is positioned is concurrently analyzed and monitored to prevent complete perforation of said inserter through the entire wall of said lumen.

20. The method of claim 10, wherein the release of agents is monitored with said fiber optic probe and controlled using feedback from said monitoring.
